(19) United States
(12) Patent Application Publication
(10) Pub. No.: US 2024/0226108 A9
(43) Pub. Date: Jul. 11, 2024

(54) TREATMENT OF BRAIN INJURIES

(71) Applicant: ASTON UNIVERSITY, West Midlands (GB)

(72) Inventors: Roslyn Mary Bill, West Midlands (GB); Stuart David Greenhill, West Midlands (GB); Luke David Southan, West Midlands (GB)

(21) Appl. No.: 18/277,718

(22) PCT Filed: Feb. 4, 2022
(86) PCT No.: PCT/GB2022/050304

§ 371 Date: Aug. 17, 2023

(30) Foreign Application Priority Data
Feb. 17, 2021 (GB) 2102247.0

(51) Int. Cl. A61K 31/5415; A61K 31/46; A61P 25/00

(52) U.S. Cl. CPC A61K 31/5415; A61K 31/46; A61P 25/00

(57) ABSTRACT

This invention relates to a method of treating damage to the central nervous system, comprising administering a pharmaceutically-effective amount of a CCR5 antagonist, and a pharmaceutically effective amount of an AQP4 antagonist, or pharmaceutically acceptable salts thereof.

This invention relates to the treatment of brain injuries, such as stroke, traumatic brain injury and vascular dementia, using the combination of an antagonist of CCR5 and an antagonist of AQP4. A combination of such antagonists is also provided.

Stroke and traumatic brain injury (TBI) are the leading causes of adult disability due to limited neurological recovery. Approximately 50-60% of patients continue to suffer motor impairments after stroke. 43% of those hospitalised for TBI suffer from long-term disability. Vascular dementia may develop after a stroke blocks an artery within the brain. Alternatively, vascular dementia can also result from other conditions that damage blood vessels and reduce circulation. This produces symptoms which may be similar to other types of dementia, such as cognitive symptoms, memory loss, problems speaking, and visuo spatial skills.

CCR5 (C—C chemokine receptor 5) has been shown to be expressed in cortical neurons after stroke. Post-stroke lockdown or administration of CCR5 antagonists have been shown to lead to early recovery of motor control and reduce cognitive decline (JOY M. T. et al, Cell 2019, 176(5), 1143-1157). Inhibition of CCR5 signalling has also been shown to enhance learning, memory and plasticity processes in hippocampal and cortical circuits. Motor recovery from CCR5 knockdown is a result of heightened plasticity in the pre-motor cortex as associated with stabilisation of dendritic spines in pre-motor cortex adjacent to the stroke sight. Up-regulation of CREB (cAMP response element-binding protein) and dual lucine zipper kinase (DLK) signalling was observed in neurons with CCR5 knockdown and formation on new connections in contralateral pre-motor cortex. Provision of the CCR5 antagonist Maraviroc produced similar effects on motor recovery post-stroke and cognitive decline post traumatic brain injury.

Central Nervous System (CNS) edema, which is swelling of the brain or spinal cord affects millions of people every year. The water channel protein aquaporin-4 (AQP4) is expressed in astrocytes and mediates water flux across the blood-brain and blood-spinal cord barriers. AQP4 cell-surface abundance has been shown to increase in response to hypoxia-induced cell swelling in a calmodulin-dependent manner. Calmodulin directly binds the AQP4 carboxyl terminus, driving AQP4 cell-surface localisation. Inhibition of calmodulin with the licenced drug trifluoroperazine (TFP) inhibited AQP4 localisation to the blood-spinal cord barrier, reduced CNS edema and led to accelerated functional recovery compared with untreated animals (Kitchen P. et al, Cell 2020, 181, 784-799).

CNS edema is caused by traumatic injuries, infection, tumour growth and stroke. These are known to increase an increased risk of neurodegenerative conditions, such as Alzheimer's disease and Parkinson's disease.

Kitchen et al showed a direct mechanistic relationship between inhibition of AQP4 function and reduction in CNS and edema. The paper demonstrates that targeting sub-cellular localisation of a membrane channel protein, rather than targeting its activity directly, produced a potential therapeutic strategy. They did this by targeting calmodulin as AQP4 required the interaction with calmodulin for sub-cellular relocalisation. Using TFP (trifluoperazine) targeted calmodulin and prevented onset of brain edema. Therefore suggesting TFP is an indirect antagonist of AQP4.

The Applicant has now realised that the mechanisms shown for targeting CCR5 and the water channel protein AQP4 are separate. They use different pathways. Accordingly, being able to target the two different pathways allows for the reduction caused by hypoxic events resulting from reduced blood flow by synergistically increasing neuronal plasticity and reducing CNS edema. The dual approach to targeting the two different pathways minimises damage suffered by brain tissue during hypoperfusion and increases the plasticity potential of preserved neurones, thereby slowing the progress and reducing the burden of vascular dementia and other such CNS damage.

Another further advantage of the dual-treatment described below, is that there are a variety of currently-prescribed CCR5 antagonists available due to their current use as entry inhibitors for HIV. Moreover, there is also a licenced anti-psychotic drug available which ablates the function of the water channel AQP4 via interaction with calmodulin. The ability to use the two types of compound which have already been characterised and proven clinically for other conditions, allows the production of a treatment for brain injury with fewer clinical risks as clinical drugs are already known for other conditions.

The invention provides a method of treating damage to the central nervous system comprising administering a pharmaceutically effective amount of a CCR5 antagonist and a pharmaceutically effected amount of an AQP4 antagonist.

A further aspect of the invention provides a CCR5 antagonist and an AQP4 antagonist for use in the treatment of damage to the central nervous system.

The CCR5 antagonist and the AQP4 antagonist may be administered together or sequentially in any order. The damage to the central nervous system may be selected from stroke, traumatic brain injury, vascular dementia or an inflammatory brain condition including dementias with an inflammatory pathology. The inflammatory brain condition may also include, for example, encephalitis. Encephalitis may be caused by a bacterial or viral infection. Viruses which are known to cause encephalitis include herpes simplex virus, Epstein Barr virus, varicella-zoster virus, enteroviruses, mosquito-borne viruses, such as West Nile and St. Louis virus, tick borne viruses such as Powassan virus, rabies virus and measles, mumps and rubella viruses. Dementias with an inflammatory pathology may include, for example, Alzheimer's Disease, Parkinson's Disease, and Fronto-temporal Dementia.

CCR5 inhibitors or antagonists are generally known in the art. They include, for example, maraviroc (sold under the brand names Selzentry and Celsentri to be used in the treatment of HIV infection). Other CCR5 inhibitors or antagonists include. Aplaviroc (also known as AK602 and GSK-873140), Cenicriviroc (also known as TAK-652 and TBR-652 developed by Takeda and Tobira Therapeutics), and Vicriviroc (also known as SCH417690 and SCH-D, developed by Schering-Plough). Other CCR5 antagonists or inhibitors include monoclonal antibodies, such as Leronlimab, developed by CytoDyn Inc.). Most typically, the CCR antagonist is Maraviroc. This has been approved by US Food and Drug Administration for the treatment of HIV infection. Details of clinically approved compounds may be found, for example, on the FDA Orange Book website or the UK NICE BNF website.

The AQP4 antagonist is typically an indirect translocation inhibitor acting on calmodulin or protein kinase A as has been shown (Kitchen P. et al. Cell 181(4), 784-799) or a direct inhibitor of AQP.4. Though use of a vasopressin antagonist is also possible (such as SR49059) as AQP4 expression has also been indicated as being reduced using such compounds (Taya K. et al, ACTA Neurochirugica. Suppl. 2008, 102, 425-9).

The calmodulin antagonist is typically trifluoperazine (TFP). However, other calmodulin inhibitors may also be used. Known inhibitors of carmodulin in the art include Loperamide, Cinchocaine, Fluphenazine, Perphenazine, Phenoxybenzamine, Promethazine, Pimozide, Aprindine, Flunarizine, Nifedipine and Chlorpromazine.

Details of clinically approved compounds may be found, for example, on the FDA Orange Book website or the UK NICE BNF website.

Compounds may be administered by any suitable means, including orally, intramuscularly, intranasally or intravenously. The inhibitors may be used in combination with one or more suitable excipients, including diluents, binders, granulating agents, glidants, lubricants or disintegrants. The compounds may be in tablet form, or alternatively, for example in the form of a syrup, suspension, emulsion or elixirs.

The inhibitors may be provided as pharmaceutically acceptable salts.

The invention also provides a combination of a CCR5 antagonist in combination with an AQP4 antagonist or pharmaceutically acceptable salts thereof. The CCR5 antagonist and AQP4 antagonist may be as described above. Pharmaceutical compositions comprising combination of a CCR5 antagonist in combination with an AQP4 antagonist or pharmaceutically acceptable salts thereof are also provided.

There may be provided mixed together, or alternatively packaged together so that they can be taken sequentially by a subject. They may be packaged together with an information sheet comprising information to a patient or doctor for the administration of the compounds in combination or sequentially.

The invention will now be described by way of example only but with reference to the following figures.

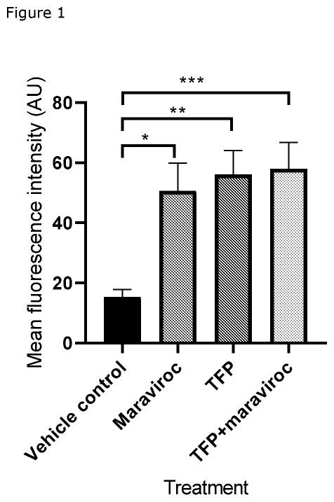

FIG. 1 shows Mean fluorescent intensity (MFI) of brain slices stained with calcein AM (acetoxymethyl). Brain slices were pre-treated for 1 hour with final concentrations of Maraviroc (50 μM), trifluoperazine (100 μM), and both drugs trifluoperazine and maraviroc against vehicle control for 1 hour, before being subjected to 30 minutes of hypoxia. Vehicle control slices were incubated in (0.125% DMSO before hypoxia was induced. Data is expressed as means±SEM of vehicle control (n=5) Maraviroc (n=5), trifluoperazine (n=5), trifluoperazine and Maraviroc (n=5). One-way ANOVA was performed p≤0.001. Kruskal-Wallis multiple comparisons test was performed (*) p≤0.05 (**) p≤0.01 (***) p≤001.

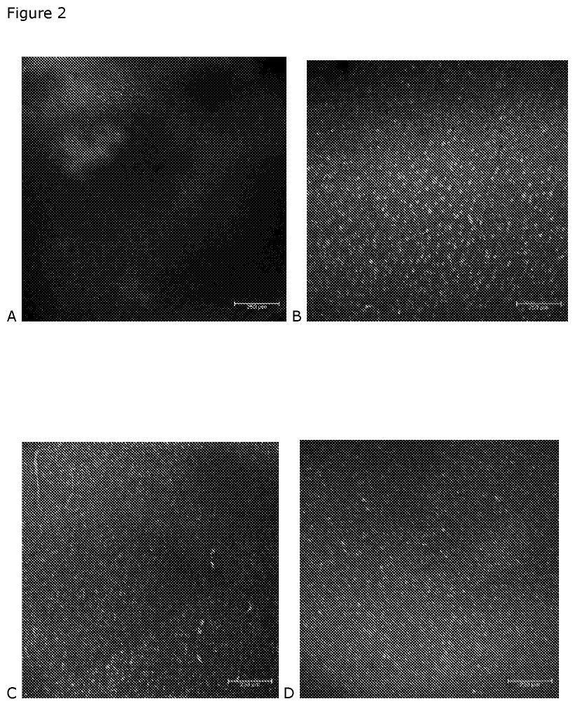

FIG. 2 shows Confocal micrographs of calcein AM (acetomethyl) stained brain slices incubated with (A—vehicle control (0.125% DMSO), (B—Trifluroperazine (100 μM) and maraviroc (50 μM) (C—Maraviroc (50 μM), (D—Trifluroperazine (100 μM) for 1 hour before 30 minutes of hypoxia was induced. Magnification (×10) and scale bar represents 250 μm.

FIG. 1 shows the effect of different compounds on hypoxia-induced brain damage. Fresh slices of rat brain tissue were incubated at 37° C. for 1 hour with control (0.125% DMSO), Maraviroc (50 μM) in DMSO, trifluoperazine (100 μM), and both drugs trifluoperazine (100 mM) and maraviroc (50 mM), prior to subjecting to hypoxia by incubating each slice under CO₂ or nitrogen for 30 minutes.

Damage to the brain tissue was determined by staining the tissue with acetoxymethyl calcein (calcein AM), available from Abcam Ltd, Cambridge, UK and Thermo Fisher, USA. The assay is a quantitative assay used to determine cell viability.

The staining of the cells under a confocal microscope is shown in FIG. 2.

The Anova and Krustal-Wallis Test results (below) show that the combination of Maraviroc and trifluoperazine improved cell viability. The improvement in the viability of the cells is expected to be improved in vivo with longer treatment of damaged tissue.

Kruskal-Wallis test
ANOVA results

| Table Analyzed | Data 4 |
|---|---|
| Kruskal-Wallis test | |
| P value | <0.0001 |
| Exact or approximate P value? | Approximate |
| P value summary | **** |
| Do the medians vary signif. (P < 0.05)? | Yes |
| Number of groups | 4 |
| Kruskal-Wallis statistic | 23.78 |
| Data summary | |
| Number of treatments (columns) | 4 |
| Number of values (total) | 60 |

Kruskal-Wallis test
Multiple comparisons
Number of families 1
Number of comparisons per family 3
Alpha 0.05

| Dunn's multiple comparisons test | Mean rank diff. | Significant? | Summary | Adjusted P Value | A-? | |
|---|---|---|---|---|---|---|
| Vehicle control vs. Maraviroc | −22.07 | Yes | ** | 0.0016 | B | Maraviroc |
| Vehicle control vs. TFP | −25.87 | Yes | *** | 0.0001 | C | TFP |
| Vehicle control vs. TFP + maraviroc | −27.13 | Yes | **** | <0.0001 | D | TFP + maraviroc |
| Test details | Mean rank 1 | Mean rank 2 | Mean rank diff. | n1 | n2 | Z |
| Vehicle control vs. Maraviroc | 11.73 | 33.80 | −22.07 | 15 | 15 | 3.460 |
| Vehicle control vs. TFP | 11.73 | 37.60 | −25.87 | 15 | 15 | 4.056 |
| Vehicle control vs. TFP + maraviroc | 11.73 | 38.87 | −27.13 | 15 | 15 | 4.255 |

1. A method of treating damage to the central nervous system, comprising administering a pharmaceutically-effective amount of a CCR5 antagonist, and a pharmaceutically effective amount of an AQP4 antagonist, or pharmaceutically acceptable salts thereof.

2. (canceled)

3. The method of claim 1, wherein the damage to the central nervous system is selected from a stroke or traumatic brain injury, vascular dementia or inflammatory brain condition.

4. The method of claim 1, wherein the CCR5 antagonist is selected from Maraviroc, Aplaviroc, Cenicriviroc, Vicriviroc and Leronlimab.

5. The method of claim 1, wherein the AQP4 antagonist is a calmodulin, Protein Kinase A or V1a-selective antagonist.

6. The method of claim 1, wherein the AQP4 antagonist is selected from Trifluoperazine, Loperamide, Cinchocaine, Fluphenazine, Perphenazine, Phenoxybenzamine, Promethazine, Pimozide, Aprindine, Flunarizine, Nifedipine and Chlorpromazine.

7. (canceled)

8. (canceled)

9. A pharmaceutical composition comprising the combination of a CCR5 antagonist in combination with an AQP4 antagonist, or pharmaceutically acceptable salts.

10. The pharmaceutical composition of claim 9, wherein the CCR5 antagonist is selected from Maraviroc, Aplaviroc, Cenicriviroc, Vicriviroc and Leronlimab.

11. The pharmaceutical composition of claim 10, wherein the CCR5 antagonist is Maraviroc.

12. The pharmaceutical composition of claim 9, wherein the AQP4 antagonist is a calmodulin, Protein Kinase A or V1a-selective antagonist.

13. The pharmaceutical composition of claim 9, wherein the AQP4 antagonist is selected from Trifluoperazine, Loperamide, Cinchocaine, Fluphenazine, Perphenazine, Phenoxybenzamine, Promethazine, Pimozide, Aprindine, Flunarizine, Nifedipine and Chlorpromazine.

14. The pharmaceutical composition of claim 13, wherein the AQP4 antagonist is Trifluoperazine.

15. The method of claim 4, wherein the CCR5 antagonist is Maraviroc.

16. The method of claim 6, wherein the AQP4 antagonist is Trifluoperazine.
